(19) United States
(12) Patent Application Publication
(10) Pub. No.: US 2021/0386271 A1
(43) Pub. Date: Dec. 16, 2021

(54) Introducer Clip for an Intubation Tube

(71) Applicants: Gabriel Peterman, Alexandria, LA (US); Taten Peterman, Alexandria, LA (US)

(72) Inventors: Gabriel Peterman, Alexandria, LA (US); Taten Peterman, Alexandria, LA (US)

(21) Appl. No.: 16/899,103

(22) Filed: Jun. 11, 2020

(51) Int. Cl. A61B 1/00; A61B 1/267; A61M 16/04

(52) U.S. Cl. CPC A61B 1/0014; A61M 16/0488; A61B 1/00137; A61B 1/267

(57) ABSTRACT

The present disclosure provides a clip for an intubation stylet or bougie onto a laryngoscope blade. A bougie attached to the clip may be easily guided into a trachea of a patient. The bougie can then be easily disconnected and subsequently used as a stylet for a breathing tube. The disclosed bougie introducer clip may be made to fit a variety of different laryngoscope blades. In preferred embodiments, the disclosed bougie clip works well with Glidescope™ laryngoscope blades. The disclosed bougie introducer clip allows for manipulation of the bougie for advancing the bougie into the airway.

### BACKGROUND OF THE DISCLOSURE

### Field of the Invention

The present invention relates to the field of introducer guides for assisting intubation procedures and intubation devices including laryngoscopes.

### Description of the Related Art

When performing medical intubations, an intubation blade is inserted into the throat. A camera can also be simultaneously used to view the inside of the throat. A bougie is then maneuvered along the intubation blade and into the trachea of the patient and subsequently used as a stylet for a breathing tube.

In some cases, it is extremely difficult to get the bougie into the patient's trachea. The process of guiding an intubation stylet into the trachea is difficult. In fact, one of the main problems with current intubation procedures is difficulty in placing the bougie. It can be difficult if not impossible on subjects who, for example, have abnormal airways, are obese, utilize a cervical spine collar, have arthritis, have mandibular fractures, have had previous cervical fusion, or are uncooperative.

An intubation stylet or bougie may be inserted into a tube guide and directed along its length into the trachea. However, in order to remove the laryngoscope and bougie once the bougie is in place, the blade must be slid back along the inserted bougie towards the oral cavity in order to separate the blade from the bougie. This can be difficult as it requires holding both the laryngoscope and the inserted bougie while withdrawing the blade, and can also be time-consuming, which is a significant disadvantage for a technique which is commonly used for medical procedures in which time may be of the essence. Current technologies do not allow for the bougie to be quickly, easily, and conveniently disconnected from the intubation blade. Current state-of-the-art products are larger and do not allow the bougie to easily detach from the blade. For example, there may be the necessity to use a breathing tube to detach the bougie from the blade.

Accordingly, there is a need in the art for mechanisms to clip an intubation stylet or bougie onto a laryngoscope blade that allows easy and direct manipulation of the intubation stylet or bougie in a patient's trachea and that may then be quickly and easily disconnected from the laryngoscope blade.

### SUMMARY OF THE INVENTION

The present disclosure provides a clip for an intubation stylet or bougie onto a laryngoscope blade. A bougie attached to the clip may be easily guided into a trachea of a patient. The bougie can then be easily disconnected and subsequently used as a stylet for a breathing tube. The disclosed bougie introducer clip may be made to fit a variety of different laryngoscope blades. In preferred embodiments, the disclosed bougie clip works well with Glidescope™ laryngoscope blades. The disclosed bougie introducer clip allows for manipulation of the bougie for advancing the bougie into the airway.

Other features and aspects will be apparent from the following detailed description, the drawings, and the claims

### BRIEF DESCRIPTION OF THE DRAWINGS

FIG. 1 shows one embodiment of an intubation stylet or bougie clip for a laryngoscope blade of the disclosure.

FIG. 2 shows a top down view of an intubation stylet clip for a laryngoscope blade of the disclosure.

FIG. 3 shows another embodiment of an intubation stylet clip for a laryngoscope blade of the disclosure.

FIG. 4A shows an embodiment of an intubation stylet clip of the disclosure attached to a Glidescope™ laryngoscope blade.

FIG. 4B shows another embodiment of an intubation stylet clip of the disclosure attached to a Glidescope™ laryngoscope blade.

FIG. 5 shows another embodiment of a clip of the disclosure.

FIG. 6 shows another embodiment of a clip of the disclosure.

FIG. 7 shows another embodiment of a clip of the disclosure.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

The present disclosure thus provides an intubation stylet or bougie clip for a laryngoscope blade that enables direct placement of a bougie into a patient's trachea. In preferred embodiments, the laryngoscope blade may be a Glidescope™ laryngoscope blade.

In embodiments the intubation stylet may comprise a circular introducer that attaches to either a laryngoscope blade or a separate clip to thread or hold an intubation stylet or bougie.

The disclosed intubation stylet or bougie clip for a laryngoscope blade also permits the ability to have some manipulation of a bougie into a patient's trachea. In embodiments, it is small and easy to remove from the laryngoscope blade handle.

The clip comprises a generally circular introducer that is connected to a clip member on one side. The clip member may comprise one or more elongation members attached at approximately right angles with an attachment extension one on side. The laryngoscope blade may then be attached between an elongation member and the attachment extension. This allows the product to be clipped onto the laryngoscope blade. The circular member may be on either side of the clip and the attachment extension may be on either side as well. In embodiments, the circular member is integrally formed with the clip.

The size of the various components of the disclosed intubation stylet clip described above can vary to adjust to the size of a particular intubation blade. The circular part is used to thread or hold the intubation stylet or bougie. The circular part is of sufficient size to allow for maneuverability of a bougie while inside the throat. A gap in the circular part allows the intubation stylet or bougie to easily detach from an intubation blade. The gap is small enough to prevent the bougie from leaving the hole without applying force; however, it is large enough to allow the bougie to detach easily while inside the throat with only a small amount of applied force. The disclosed clip circular member may then be adjusted to be attached close to the center of an intubation blade, yet far enough away to allow a camera a clear view of the trachea.

The disclosed intubation stylet clip for a laryngoscope blade can be made of any sturdy bio-safe plastic. The size of the clip can be adjusted as needed for the size of a laryngoscope blade. Laryngoscope blade sizes vary and clip sizes compatible with known and used laryngoscope blade sizes in the art are contemplated. A sturdy bio-safe plastic may bend slightly with pressure, but the product is generally made to hold its shape.

It is noted that bougies come in different sizes, and the introducer on the disclosed clip can easily be adjusted for any size bougie. Three main bougies are used for intubation. One is a standard adult bougie. A second is a special hollow bougie that can deliver oxygen to a patient. A third is a pediatric bougie. An adult bougie is the largest bougie, and the pediatric bougie is the smallest bougie.

For an adult bougie, the maximum diameter of the center hole of the circular introducer may be about 0.3 inches. For a pediatric bougie, the minimum diameter of the center hole may be about 0.2 inches.

The gap of the circular introducer may have a range of about 0.13 inches to about 0.185 inches depending on the bougie size that the introducer is designed for. The circular member may have an extended length of about 0.5 inches. These dimensions provide for bougie maneuverability while also allowing control over it.

The circular introducer member may have a constant diameter of about 0.4 inches. Because the size of the center cut hole does not necessarily affect the total diameter of the circular member, the circular member introducer may have a thickness range of about 0.05 inches to about 0.1 inches depending on the required size of the center hole.

In one example, most edge curves on the clip and the circular introducer may have a radius of about 0.05 inches. However, the attachment extension that runs underneath the blade may have an end curve with a radius of about 0.15 inches, and the side attachment extension that runs along the flat part of the intubation blade may have an end curve with a radius of about 0.35 inches. The clip attachment may have a constant thickness of about 0.1 inches.

The other measurements for the introducer portion of the clip may be constant for the various bougies. The dimension ranges for the attachment portion of the clip is more complicated as laryngoscope blades come in different shapes and sizes. Generally speaking, the dimensions for the attachment portion can be varied to fit a given laryngoscope blade.

In a preferred embodiment, the disclosed clip may be used with Glidescope™ video intubation blades. Glidescope™ intubation blades include clear plastic GVL 3 and GVL 4 models. The disclosed product works well with these blades. These blades slightly differ in shape, so there are variations in the clip shape for each blade. The introducer portion of the clip can remain the same in all cases.

For Glidescope™ intubation blades, because the GVL 4 model is the largest, the dimensions for its corresponding introducer clip may be considered maximum Glidescope™ dimensions. Because the GVL 0 model is the smallest, the dimensions for its corresponding introducer can be considered the minimum Glidescope™ dimensions. For the introducer clip, the maximum attachment total length of the clip is about 1.225 inches, and the minimum attachment total length needs to be about 0.554 inches for the Glidescope™ models.

These total attachment lengths ignore any extra length added by the circular introducer because the circular introducer's position may be adjusted to fit the shape of the laryngoscope blade. The maximum attachment inside length is about 1.025 inches, and the minimum attachment inside length is about 0.354 inches. The attachment extension that runs underneath the blade has a maximum length of about 0.4 inches. This extension's size can decrease to 0 inches depending on the shape of the intubation blade. Furthermore, this extension is placed at a minimum of about 0.14 inches from the underside of the top of the clip. This extension can be placed at a maximum of about 0.5 inches. All other measurements may be constant.

Dimensions for other models (non-Glidescope™) may be arrived at accordingly. It is generally expected that the relevant dimensions will be similar to the above disclosed GVL Glidescope™ models and may fall within the above ranges. For a given model, it is understood that the above disclosure and ranges would generally permit one of ordinary skill to make the variations necessary to fit the clip to a particular laryngoscope blade.

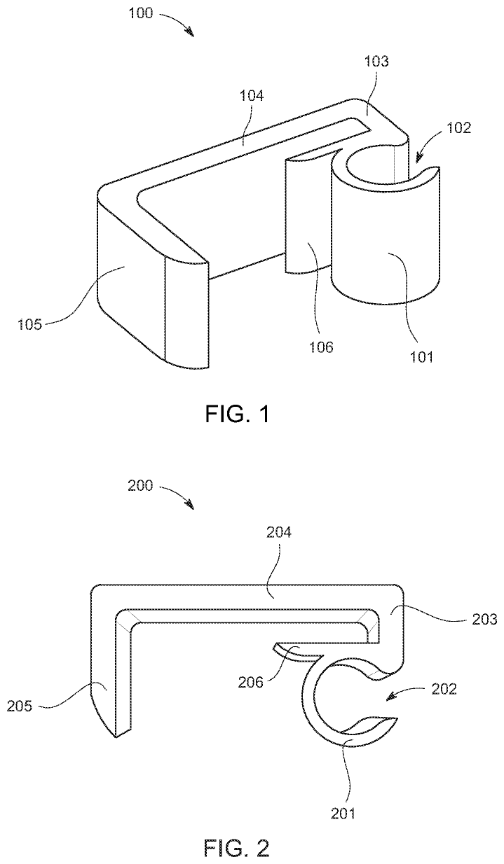

FIG. 1 shows one embodiment of an intubation stylet clip 100 for a laryngoscope blade of the disclosure. Clip 100 includes circular member 101 with gap 102, elongation members 103, 104, and 105 and attachment extension 106.

FIG. 2 shows a top down view of an intubation stylet clip 200 for a laryngoscope blade of the disclosure. FIG. 2 shows elongation members 203, 204, and 205, circular member 201 with gap 202, and attachment extension 206.

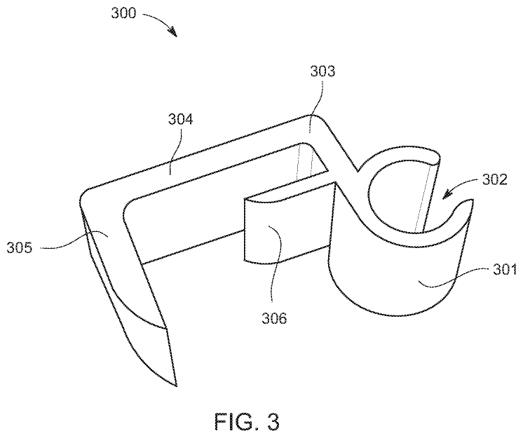

FIG. 3 shows another embodiment of an intubation stylet clip 300 for a laryngoscope blade of the disclosure. FIG. 3 shows elongation members 303, 304, and 305, attachment extension 306, and circular member 301 with gap 302.

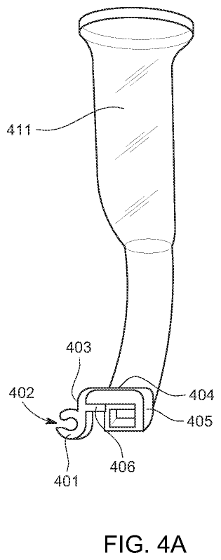

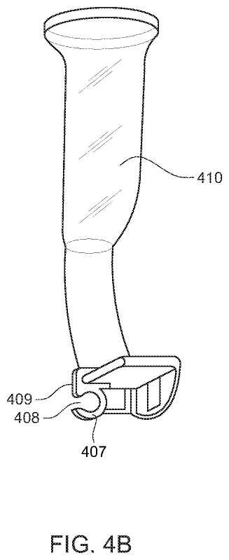

FIG. 4A shows an embodiment of an intubation stylet clip of the disclosure attached to a laryngoscope blade 411. FIG. 4A shows elongation members 403, 404, and 405, attachment extension 406, and circular member 401 with gap 402.

FIG. 4B shows another embodiment of an intubation stylet clip of the disclosure attached to a laryngoscope blade 410. FIG. 4B shows elongation member 409, and circular member 407 with gap 408.

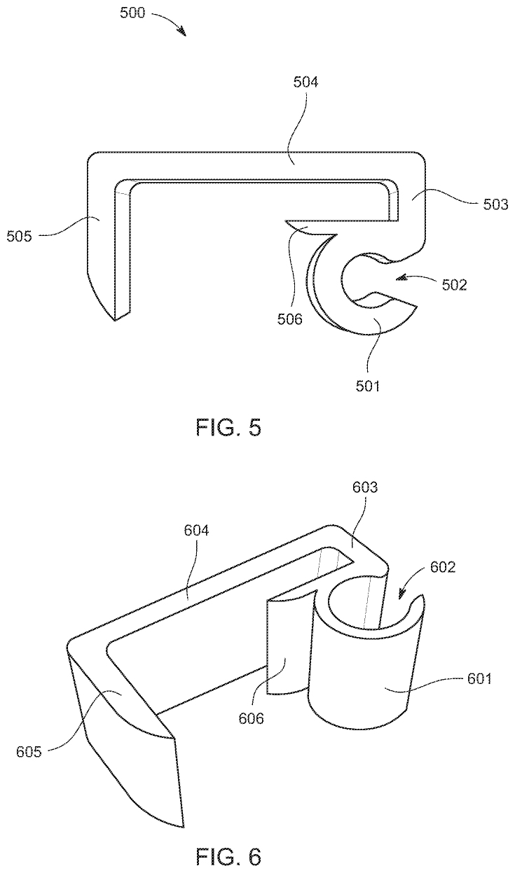

FIG. 5 shows another embodiment of an intubation stylet clip 500 for a laryngoscope blade of the disclosure which may be suitable for a pediatric bougie. FIG. 5 shows elongation members 503, 504, and 505, attachment extension 506, and circular member 501 with gap 502.

FIG. 6 shows another view of an intubation stylet clip for a laryngoscope blade of the disclosure. FIG. 6 shows elongation members 603, 604, and 605, attachment extension 606, and circular member 601 with gap 602.

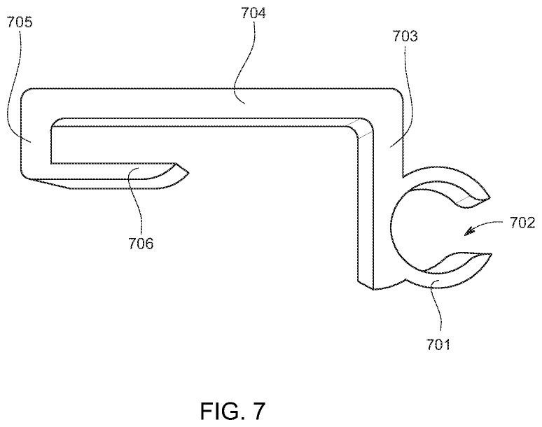

FIG. 7 shows another view of an intubation stylet clip for a laryngoscope blade of the disclosure. FIG. 7 shows elongation members 703, 704, and 705, attachment extension 706, and circular member 701 with gap 702.

### Examples

Glidescope™ intubation blades are widely used across many hospitals. In preferred embodiments, the disclosed clip connects to a standard Glidescope™ intubation blade.

In this example, the clip is made from ABS (acrylonitrile butadiene styrene). The circular member is a 0.4 inch circle with a 0.3 inch hole at or near the center. The gap was created from a 0.185 inch diameter hole created along an outer side of the circular member. 0.05 inch fillets were then cut along the inside opening. In this example, the clip was made as a single piece on a 3D printer. This clip was successfully tested on an intubation training dummy.

In a second example, the dimensions relate to the GVL 4 Glidescope™ model which is commonly used. The total length of the GVL 4 clip attachment is 1.225 inches with an inside length of 1.025 inches. For the GVL 4 clip, the circular introducer is placed directly under a 0.4-inch bottom attachment extension.

While this disclosure includes specific examples, it will be apparent after an understanding of the disclosure of this application has been attained that various changes in form and details may be made in these examples without departing from the spirit and scope of the claims and their equivalents.

We claim:

1. An intubation stylet for a laryngoscope blade comprising:
a circular introducer wherein the circular member comprises a gap.

2. An intubation stylet for a laryngoscope blade of claim 1 further comprising:
one or more elongation members connected to form a clip that attaches to a laryngoscope blade;
the circular introducer comprising a gap being connected to the clip;
and wherein the intubation stylet blade clip is attachable and detachable from the laryngoscope blade.

3. The intubation stylet for a laryngoscope blade of claim 2, wherein the circular introducer comprises an inner diameter ranging from about 0.2 inches to about 0.3 inches.

4. The intubation stylet for a laryngoscope blade of claim 2, wherein the circular introducer gap has a width of about 0.13 inches to about 0.185 inches.

5. The intubation stylet clip for a laryngoscope blade of claim 2, further comprising an attachment extension connected to an elongation member that holds the clip onto the laryngoscope blade.

6. The intubation stylet clip for a laryngoscope blade of claim 5, wherein the attachment extension length ranges from about 0.3 inches to about 1.3 inches.

7. The intubation stylet clip for a laryngoscope blade of claim 2, wherein the circular introducer is integrally formed with the one or more elongation members.

8. The intubation stylet clip for a laryngoscope blade of claim 2, wherein the laryngoscope blade is a Glidescope™ intubation blade.

9. The intubation stylet clip for a laryngoscope blade of claim 2 wherein there are three elongation members connected at approximately right angles.

10. The intubation stylet clip for a laryngoscope blade of claim 9, further including an attachment extension connected to an elongation member, wherein the circular introducer, the attachment member, and the elongation members are integrally formed.
